(19) United States
(12) Patent Application Publication
(10) Pub. No.: US 2012/0020930 A1
(43) Pub. Date: Jan. 26, 2012

(54) METHODS FOR THE PREPARATION OF ADIPOSE DERIVED STEM CELLS AND UTILIZING SAID CELLS IN THE TREATMENT OF DISEASES

(75) Inventors: Dirk Buescher, Tres Cantos - Madrid (ES); Eleuterio Lombardo, Tres Cantos - Madrid (ES)

(73) Assignee: CELLERIX, S.A., Tres Cantos - Madrid (ES)

(21) Appl. No.: 13/132,625

(22) PCT Filed: Dec. 2, 2009
(86) PCT No.: PCT/EP2009/066198

§ 371 Date: Sep. 29, 2011

(30) Foreign Application Priority Data
Dec. 3, 2008 (EP) 08382070.4

(51) Int. Cl. A61K 35/12; A61P 29/00; A61P 37/08; A61P 37/00; C12N 5/0775; A61P 43/00

(52) U.S. Cl. 424/93.7; 435/375

(57) ABSTRACT

The present invention provides a method for the preparation of adipose derived stem cells for use in cellular therapy. In further aspects the present invention provides cells produced by the method of the invention, pharmaceutical compositions and kits suitable for use in treatment of patients.

### FIELD OF THE INVENTION

The present invention relates to methods for the preparation of adipose derived stem cells and the utilization of said cells in the treatment of diseases.

### BACKGROUND OF THE INVENTION

STEM CELL THERAPY Mesenchymal stem cells (MSCs) are multipotent adult stem cells capable of differentiation into mesenchymal-type cells (adipocytes, osteoblasts and chondrocytes), and also myocytes, neurons, endothelial cells, astrocytes and epithelial cells. Although first reported in the normal adult bone marrow (BM-MSC), MSCs can also be obtained from other sources, such as umbilical cord blood, peripheral blood and adipose tissue.

The adipose tissue is a source of MSCs referred to as human adipose-derived mesenchymal stem cells (hASC), which can be isolated from liposuctioned fat tissue and expanded over a long time in culture. hASCs share some features with their counterpart in marrow, such as their differentiation potential, low immunogenicity and the ability to suppress immune responses. Recent studies comparing both cell types have reported differences at transcriptional and proteomic levels, suggesting that hASC and BM-MSC, while sharing similarities, are in fact quite different. The specific mechanisms underlying hASCs-mediated immunosuppression have so far been poorly studied. Recently, it has been reported that hASCs may inhibit lymphocyte proliferation by a mechanism that requires, at least in part, the release of PGE2. However, these studies did not provide information regarding (i) other cellular or soluble factors involved in the mechanism of immunosuppression, (ii) the immunosuppressive effect on isolated T cell subsets, or (iii) the phenotypic changes in both hASCs and PBMCs upon co-culture.

These biological abilities make MSCs, including hASCs, an interesting tool for cellular therapy and regeneration. This is further supported by studies showing that BM-MSCs alleviate allograft rejection, graft-versus-host disease, experimental autoimmune encephalomyelitis, collagen-induced arthritis and autoimmune myocarditis. Moreover, it has been recently reported that mouse ASCs (mASCs) were very efficient in protecting against graft-versus-host disease after allogeneic transplantation in an in vivo mouse model. In addition, MSCs are being used in several clinical trials with a focus on their immunomodulatory and anti-inflammatory capacities. Adipose derived stem cells therapies appear promising in the treatment of a wide variety of diseases ranging from tissue regeneration to immune and/or inflammatory diseases. Although methods are available for treating these diseases, many current therapies provide less than adequate results, and carry the risk of significant side effects. Among new emergent therapeutic strategies, those based on cell therapy appear to constitute a potentially useful tool for treating a large number of diseases. Thus, a great effort is currently being made by researchers in order to achieve said aim.

AUTOIMMUNE DISEASES Autoimmune diseases are caused when the body's immune system, which is meant to defend the body against bacteria, viruses, and any other foreign product, malfunctions and produces a pathological response against healthy tissue, cells and organs.

T cells and macrophages provide beneficial protection, but can also produce harmful or deadly immunological responses. Autoimmune diseases can be organ specific or systemic and are provoked by different pathogenic mechanisms. Systemic autoimmune diseases involve polyclonal B cell activation and abnormalities of immunoregulatory T cells, T cell receptors and MHC genes.

Examples of organ specific autoimmune diseases are diabetes, hyperthyroidism, autoimmune adrenal insufficiency, pure red cell anemia, multiple sclerosis and rheumatic carditis. Representative systemic autoimmune diseases include systemic lupus erythematosus, chronic inflammation, Sjogren's syndrome, polymyositis, dermatomyositis and scleroderina

Current treatment of autoimmune diseases involves administering immunosuppressive agents such as cortisone, aspirin derivatives, hydroxychloroquine, methotrexate, azathioprine and cyclophosphamide or combinations thereof. The dilemma faced when administering immunosuppressive agents, however, is the more effectively the autoimmune disease is treated, the more defenseless the patient is left to attack from infections, and also the more susceptible for developing tumors. Thus, there is a great need for new therapies for the treatment of autoimmune diseases.

INFLAMMATORY DISORDERS Inflammation is a process by which the body's white blood cells and secreted factors protect our bodies from infection by foreign substances, such as bacteria and viruses and is a common process in autoimmune diseases. Secreted factors known as cytokines and prostaglandins control this process, and are released in an ordered and self-limiting cascade into the blood or affected tissues. In general, the current treatments for chronic inflammatory disorders have a very limited efficiency, and many of them have a high incidence of side effects or cannot completely prevent disease progression So far, no treatment is ideal, and there is no cure for these type of pathologies. Thus, there is a great need for new therapies for the treatment of inflammatory disorders.

### BRIEF DESCRIPTION OF THE FIGURES

FIG. 1 depicts the number of migrating cells (Y-axis) at varying level of Diprotin A (Ile-Pro-Ile) -an inhibitor of DPP-4 (X-axis), as described in Example 1. As can be seen from FIG. 1 increasing levels of Diprotin A result in increased migration of cells.

### SUMMARY OF THE INVENTION

The present invention provides a method for the preparation of adipose derived stem cells by exposure to a CD26 antagonist or inhibitor, resulting in the enhanced migratory capacity of said adipose derived stem cells. In further aspects the present invention provides cells produced by the method of the invention, pharmaceutical compositions and kits suitable for use in treatment of patients.

Definitions

In order to facilitate the understanding of the present description, the meaning of some terms and expressions in the context of the invention will be explained below. Further definitions will be included along the description as necessary.

As used herein the term “DPP-4” shall be taken to mean the transmembrane glycoprotein Dipeptidyl peptidase IV (also known as CD26 and DPP IV) and shall be taken to include all variants thereof having the activity of inhibiting CXCR4 mediated chemotaxis.

The term “DPP-4 antagonist or inhibitor” shall be taken to mean an agent that inhibits DPP-4 inhibition of CXCR4 mediated chemotaxis.

The term “CXCR-4” shall be taken to mean the cell surface chemokine receptor also known as “fusin”.

The term “allogeneic” as used herein shall be taken to mean from different individuals of the same species. Two or more individuals are said to be allogeneic to one another when the genes at one or more loci are not identical.

The term “autologous” as used herein shall be taken to mean from the same individual.

The term “autoimmune disease” refers to a condition in a subject characterized by cellular, tissue and/or organ injury caused by an immunological reaction of the subject to its own cells, tissues and/or organs. Illustrative, non-limiting examples of autoimmune diseases which can be treated with the immunomodulatory cells of the invention include alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sjogren's syndrome, Good pasture's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, Anti-Glomerular Basement Membrane Disease, Antiphospholipid Syndrome, Autoimmune Diseases of the Nervous System, Familial Mediterranean Fever, Lambert-Eaton Myasthenic Syndrome, Sympathetic Ophthalmia, Polyendocrinopathies, Psoriasis etc.

The term “ inflammatory disease” refers to a condition in a subject characterized by inflammation, e g, chronic inflammation Illustrative, non-limiting examples of inflammatory disorders include, but are not limited to, Celiac Disease, rheumatoid arthritis (RA), Inflammatory Bowel Disease (IBD), asthma, encephalitis, chronic obstructive pulmonary disease (COPD), inflammatory osteolysis, allergic disorders, septic shock, pulmonary fibrosis (e g, idiopathic pulmonary fibrosis), inflammatory vacultides (e g, polyarteritis nodosa, Wegner's granulomatosis, Takayasu's arteritis, temporal arteritis, and lymphomatoid granulomatosus), post-traumatic vascular angioplasty (e g, restenosis after angioplasty), undifferentiated spondyloarthropathy, undifferentiated arthropathy, arthritis, inflammatory osteolysis, chronic hepatitis, and chronic inflammation resulting from chronic viral or bacteria infections.

The term “subject” refers to an animal, preferably a mammal including a non-primate (e g, a cow, pig, horse, cat, dog, rat, or mouse) and a primate (e g, a monkey, or a human). In a preferred embodiment, the subject is a human.

As used herein, “negative” or “−” as used with respect to cell surface markers shall be taken to mean that mean that, in a cell population, less than 20%, 10%, preferably 9%, 8%, 7%, 6%, 5%, 4%, 3%, 2%, 1% or none of the cells express said marker. Expression of cell surface markers may be determined for example by means of flow cytometry for a specific cell surface marker using conventional methods and apparatus (for example a Becton Dickinson FACS Calibur system used with commercially available antibodies and standard protocols known in the art).

As used herein the term adipose stem cell (also referred to herein as “ASC”) shall be taken to mean a multipotent cell type originally derived from adipose tissue. The term “stem cell” shall be taken to mean a cell that, by successive divisions, can give rise to specialised cells. Multipotent stem cells can give rise to multiple types of specialized cells.

As used herein, the expression “significant expression” or its equivalent terms “positive” and “+” when used in regard to a cell surface marker shall be taken to mean that, in a cell population, more than 20%, preferably, 30%, 40%, 50%, 60%, 70%, 80%, 90% or all of the cells express said marker.

Expression of cell surface markers may be determined for example by means of flow cytometry for a specific cell surface marker using conventional methods and apparatus (for example the Becton Dickinson FACS Calibur system system used with commercially available antibodies and standard protocols known in the art) that show a signal for a specific cell surface marker in flow cytometry above the background signal using conventional methods and apparatus (for example a Becton Dickinson FACS Calibur system used with commercially available antibodies and standard protocols known in the art). The background signal is defined as the signal intensity given by a non-specific antibody of the same isotype as the specific antibody used to detect each surface marker in conventional FACS analysis. For a marker to be considered positive the specific signal observed is stronger than 20%, preferably, 30%, 40%, 50%, 60%, 70%, 80%, 90%, 500%, 1000%, 5000%, 10000% or above, than the background signal intensity using conventional methods and apparatus (for example a Becton Dickinson FACS Calibur system used with commercially available antibodies and standard protocols known in the art).

As used herein, the terms “treat”, “treatment” and “treating” when used directly in reference to a patient or subject shall be taken to mean the amelioration of one or more symptoms associated with a disorder including, but not limited to, an inflammatory disorder, an autoimmune disease or an immunologically mediated disease including rejection of transplanted organs and tissues, wherein said amelioration results from the administration of the immunomodulatory cells of the invention, or a pharmaceutical composition comprising thereof, to a subject in need of said treatment.

### DETAILED DESCRIPTION OF THE INVENTION

The present invention provides methods for the preparation of adipose derived stem cells (hereinafter referred to as ASC) for use in therapy. ASC prepared according to the method of the invention present increased mobility to in vivo locations in need of cellular therapy, and maintains the presence of the said ASC at said locations thereby increasing the efficacy thereof. Accordingly the present invention also provides pharmaceutical compositions, methods for the preparation or administration thereof, kits and uses of said aforementioned in therapy.

METHOD OF THE INVENTION In one aspect the present invention provides a method for the ex-vivo preparation of adipose derived stem cells (ASC) whereby said ASC are exposed to a DPP-4 antagonist or inhibitor. Preferably said ASC are exposed to a DPP-4 antagonist or inhibitor in an amount effective to inhibit DPP-4 activity. Said effective amount will depend on multiple factors.

In one embodiment of the method said antagonist or inhibitor is selected from the group consisting of Aminomethylpyridine; NVP DPP728; PSN9301; Isoleucine thiazolidide; Denagliptin; Sitagliptin; Vildagliptin; Saxagliptin; Alogliptin; Diprotin A. In a particularly preferred embodiment of the method said antagonist or inhibitor is Diprotin A. in one embodiment wherein said DPP-4 antagonist or inhibitor is Diprotin A said effective amount is at a concentration of between 1 and 100 mM.

With respect to the intended recipient of the therapy, the ASC used in the method of the present invention may be of either allogeneic (donor) or autologous (patient or subject) origin. In one embodiment of the method said ASC are of allogeneic origin.

The ASC may be derived from adipose tissue of any suitable origin, but is most preferably human in origin. It is preferred that said cells are obtained from non-pathological mammalian sources, preferably post-natal (e.g. rodent; primate), however particularly preferred are subcutaneous adipose tissue or organ associated adipose tissue (for example but not limited to adipose associated with the heart, liver, kidneys or pancreas).

The ASC are preferably characterized in that (i) they do not express markers specific for APCs; (ii) they do not express IDO constitutively; (iii) they express IDO upon stimulation with IFN-γ; and (iv) they present capacity to be differentiated into at least two cell lineages.

ASC Markers The ASC are preferably negative for at least one, two, three, four or preferably all of the following markers CD11b, CD11c, CD14, CD45, and HLAII, which are specific markers for APCs lineages.

Moreover, the ASC are preferably negative for at least one, two of, or preferably all of the following cell surface markers: CD31, CD34 and CD133.

As used herein, “negative” with respect to cell surface markers means that, in a cell population comprising the ASC, less than 10%, preferably 9%, 8%, 7%, 6%, 5%, 4%, 3%, 2%, 1% or none of the cells show a signal for a specific cell surface marker in flow cytometry above the background signal, using conventional methods and apparatus (for example a Beckman Coulter Epics XL FACS system used with commercially available antibodies and standard protocols known in the art). In a particular embodiment, the ASC are characterised in that they express at least one, two, three, four, of or preferably all of the following cell surface markers: CD9, CD44, CD54, CD90 and CD105; i.e., the ASC are positive for at least one, two, three, four of and preferably all said cell surface markers (CD9, CD44, CD54, CD90 and CD105). Preferably, the ASC are characterised in that they have significant expression levels of at least one, two, three, four, of and preferably all of said cell surface markers (CD9, CD44, CD54, CD90 and CD105). As used herein, the expression “significant expression” means that, in a cell population comprising the ASC, more than 10%, preferably 20%, 30%, 40%, 50%, 60%, 70%, 80%, 90% or all of the cells show a signal for a specific cell surface marker in flow cytometry above the background signal using conventional methods and apparatus (for example a Beckman Coulter Epics XL FACS system used with commercially available antibodies and standard protocols known in the art). The background signal is defined as the signal intensity given by a non-specific antibody of the same isotype as the specific antibody used to detect each surface marker in conventional FACS analysis. Thus for a marker to be considered positive the specific signal observed is stronger than 10%, preferably 20%, 30%, 40%, 50%, 60%, 70%, 80%, 90%, 500%, 1000%, 5000%, 10000% or above, than the background signal intensity using conventional methods and apparatus (for example a Beckman Coulter Epics XL FACS system used with commercially available antibodies and standard protocols known in the art).

Optionally, the ASC are also negative for the cell surface marker CD106 (VCAM-1). Examples of such cells are certain populations of adipose tissue-derived stromal stem cells as described herein.

Commercially available and known monoclonal antibodies against said cell-surface markers (e.g., cellular receptors and transmembrane proteins) can be used to identify the ASC.

Expression of IDO The ASC as used in the present invention preferably do not express IDO constitutively, but they express IDO upon stimulation with IFN-γ. Experiments Said cells, upon stimulation with other pro-inflammatory mediators such us interleukin-1 (IL-1) used at a concentration of 3 ng/ml, tumour necrosis factor-alphaTNF-α) used at a concentration of 50 ng/mi, or the endotoxin LPS used at a concentration of 100 ng/ml, did not induce IDO expression, as measured by conventional RT-PCR and Western Blot analysis. Stimulation with IFN-γ for example at 3 ng/ml or higher can also induce expression of HLAII in the ASC to give a positive signal as defined herein for a cell surface marker. Said expression can be detected by those skilled in the art using any known technique that allows the detection of the expression of specific proteins. Preferably, said techniques are cell cytometry techniques.

ASC Differentiation The ASC as used in the method of the present invention present the capacity to proliferate and be differentiated into at least two, more preferably three, four, five, six, seven or more cell lineages. Illustrative, non-limiting examples of cell lineages in which the ASC can be differentiated include osteocytes, adipocytes, chondrocytes, tenocytes, myocytes, cardiomyocytes, hematopoietic-supporting stromal cells, endothelial cells, neurons, astrocytes, and hepatocytes.

ASC can proliferate and differentiate into cells of other lineages by conventional methods. Methods of identifying and subsequently isolating differentiated cells from their undifferentiated counterparts can be also carried out by methods well known in the art.

The ASC are also capable of being expanded ex vivo. That is, after isolation, the ASC can be maintained and allowed to proliferate ex vivo in culture medium. Such medium is composed of, for example, Dulbecco's Modified Eagle's Medium (DMEM), with antibiotics (for example, 100 units/ml Penicillin and 100 □g/ml Streptomycin) or without antibiotics, and 2 mM glutamine, and supplemented with 2-20% fetal bovine serum (FBS). It is within the skill of one in the art to modify or modulate concentrations of media and/or media supplements as necessary for the cells used. Sera often contain cellular and non-cellular factors and components that are necessary for viability and expansion. Examples of sera include FBS, bovine serum (BS), calf serum (CS), fetal calf serum (FCS), newborn calf serum (NCS), goat serum (GS), horse serum (HS), porcine serum, sheep serum, rabbit serum, rat serum (RS), etc. Also contemplated is, if the ASC are of human origin, supplementation of cell culture medium with a human serum, preferably of autologous origin. It is understood that sera can be heat-inactivated at 55-65° C. if deemed necessary to inactivate components of the complement cascade. Modulation of serum concentrations, withdrawal of serum from the culture medium can also be used to promote survival of one or more desired cell types. Preferably, ASC will benefit from FBS concentrations of about 2% to about 25%. In another embodiment, the ASC can be expanded in a culture medium of definite composition, in which the serum is replaced by a combination of serum albumin, serum transferrin, selenium, and recombinant proteins including but not limited to: insulin, platelet-derived growth factor (PDGF), and basic fibroblast growth factor (bFGF) as known in the art.

Many cell culture media already contain amino acids; however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, and the like.

Antimicrobial agents are also typically used in cell culture to mitigate bacterial, mycoplasmal, and fungal contamination. Typically, antibiotics or anti-mycotic compounds used are mixtures of penicillin/streptomycin, but can also include, but are not limited to amphotericin (Fungizone®), ampicillin, gentamicin, bleomycin, hygromacin, kanamycin, mitomycin, etc.

Hormones can also be advantageously used in cell culture and include, but are not limited to, D-aldosterone, diethylstilbestrol (DES), dexamethasone, b-estradiol, hydrocortisone, insulin, prolactin, progesterone, somatostatin/human growth hormone (HGH), etc.

The maintenance conditions of the ASC can also contain cellular factors that allow cells to remain in an undifferentiated form. It is apparent to those skilled in the art that prior to differentiation, supplements that inhibit cell differentiation must be removed from the culture medium. It is also apparent that not all cells will require these factors. In fact, these factors may elicit unwanted effects, depending on the cell type.

Method for isolating ASC Methods for the isolation of ASC are known in the art, and any suitable method may be used. In one embodiment this may comprise the steps of:
(i) preparing a cell suspension from a sample of adipose;
(ii) recovering the cells from said cell suspension;
(iii) incubating said cells in a suitable cell culture medium on a solid surface under conditions which allow cells to adhere to the solid surface and proliferate;
(iv) washing said solid surface after incubation to remove non-adhered cells;
(v) selecting the cells which after being passaged at least twice in such medium remain adhered to said solid surface; and
(vi) confirming that the selected cell population presents the phenotype of interest.

As used herein, the term “solid surface” refers to any material upon which the ASC can adhere. In a particular embodiment said material is a plastic material treated to promote the adhesion of mammalian cells to its surface, for example commercially available polystyrene plates optionally coated with poly-D-Lysine or other reagents.

Steps (i)-(vi) can be carried out by conventional techniques known by those skilled in the art. Briefly, the ASC can be obtained by conventional means from any suitable source of connective tissue from any suitable animal as discussed above. Typically, human adipose cells are obtained from living donors, using well-recognized protocols such as surgical or suction lipectomy. Indeed, as liposuction procedures are so common, liposuction effluent is a particularly preferred source from which the ASC can be derived. Thus, in a particular embodiment, the ASC are from the stromal fraction of human adipose tissue obtained by liposuction. In another particular embodiment, the ASC are from human hyaline articular cartilage obtained by arthroscopic techniques. In another particular embodiment, the ASC are from human skin obtained by biopsy techniques.

The tissue is, preferably, washed before being processed to separate the ASC from the remainder of the material. In one commonly used protocol, the sample of tissue is washed with physiologically-compatible saline solution (e.g., phosphate buffered saline (PBS)) and then vigorously agitated and left to settle, a step that removes loose matter (e.g., damaged tissue, blood, erythrocytes, etc) from the tissue. Thus, the washing and settling steps are generally repeated until the supernatant is relatively clear of debris. The remaining cells generally will be present in clumps of various sizes, and the protocol proceeds using steps gauged to degrade the gross structure while minimizing damage to the cells themselves. One method of achieving this end is to treat the washed lumps of cells with an enzyme that weakens or destroys bonds between cells (e.g., collagenase, dispase, trypsin, etc.). The amount and duration of such enzymatic treatment will vary, depending on the conditions employed, but the use of such enzymes is generally known in the art. Alternatively or in conjunction with such enzymatic treatment, the lumps of cells can be degraded using other treatments, such as mechanical agitation, sonic energy, thermal energy, etc. If degradation is accomplished by enzymatic methods, it is desirable to neutralize the enzyme following a suitable period, to minimize deleterious effects on the cells.

The degradation step typically produces a slurry or suspension of aggregated cells and a fluid fraction containing generally free stromal cells (e.g., red blood cells, smooth muscle cells, endothelial cells, fibroblast cells, and stem cells). The next stage in the separation process is to separate the aggregated cells from the ASC. This can be accomplished by centrifugation, which forces the cells into a pellet covered by a supernatant. The supernatant then can be discarded and the pellet suspended in a physiologically-compatible fluid. Moreover, the suspended cells typically include erythrocytes, and in most protocols it is desirable to lyse them. Methods for selectively lysing erythrocytes are known in the art, and any suitable protocol can be employed (e.g., incubation in a hyper -or hypotonic medium, by lysis using ammonium chloride, etc.). Of course, if the erythrocytes are lysed, the remaining cells should then be separated from the lysate, for example by filtration, sedimentation, or density fractionation.

Regardless of whether the erythrocytes are lysed, the suspended cells can be washed, re-centrifuged, and resuspended one or more successive times to achieve greater purity. Alternatively, the cells can be separated on the basis of cell surface marker profile or on the basis of cell size and granularity.

Following the final isolation and re-suspension, the cells can be cultured and, if desired, assayed for number and viability to assess the yield. Preferably, the cells will be cultured without differentiation, on a solid surface, using a suitable cell culture media, at the appropriate cell densities and culture conditions. Thus, in a particular embodiment, cells are cultured without differentiation on a solid surface, usually made of a plastic material, such as Petri dishes or cell culture flasks, in the presence of a suitable cell culture medium [e.g., DMEM, typically supplemented with 5-15% (e.g., 10%) of a suitable serum, such as fetal bovine serum or human serum], and incubated under conditions which allow cells to adhere to the solid surface and proliferate. After incubation, cells are washed in order to remove non-adhered cells and cell fragments. The cells are maintained in culture in the same medium and under the same conditions until they reach the adequate confluence, typically, about 70%, about 80% or about 90% cell confluence, with replacement of the cell culture medium when necessary. After reaching the desired cell confluence, the cells can be expanded by means of consecutive passages using a detachment agent such as trypsin and seeding onto a new cell culture surface at an appropriate cell density (usually 2,000-10,000 cells/cm²). Thus, cells are then passaged at least two times in such medium without differentiating, while still retaining their developmental phenotype, and more preferably, the cells can be passaged at least 10 times (e.g., at least 15 times or even at least 20 times) without losing developmental phenotype. Typically, the cells are plated at a desired density such as between about 100 cells/cm² to about 100,000 cells/cm² (such as about 500 cells/cm² to about 50,000 cells/cm², or, more particularly, between about 1,000 cells/cm² to about 20,000 cells/cm²). If plated at lower densities (e.g., about 300 cells/cm²), the cells can be more easily clonally isolated. For example, after a few days, cells plated at such densities will proliferate into an homogeneous population. In a particular embodiment, the cell density is between 2,000-10,000 cells/cm².

Cells which remain adhered to the solid surface after such treatment comprising at least two passages are selected and the phenotype of interest is analyzed by conventional methods in order to confirm the identity of the ASC as will be mentioned below. Cells which remain adhered to the solid surface after the first passage are from heterogeneous origin; therefore, said cells must be subjected to at least another passage. As a result of the above method, a homogeneous cell population having the phenotype of interest is obtained. The adhesion of cells to the solid surface after at least two passages constitutes a preferred embodiment of the invention for selecting the ASC. Confirmation of the phenotype of interest can be carried out by using conventional means.

Preferably said expansion is carried out by duplication or triplication of said population at least 1, at least 2, at least 3, at least 4, at least 5, at least 10, at least 15 or at least 20 times. In a further embodiment said expansion is carried over at least 1, at least 2, at least 3, at least 4, at least 5, at least 10, at least 15 or at least 20 passages.

Cell-surface markers can be identified by any suitable conventional technique, usually based on a positive/negative selection; for example, monoclonal antibodies against cell-surface markers, whose presence/absence in the cells is to be confirmed, can be used; although other techniques can also be used. Thus, in a particular embodiment, monoclonal antibodies against one, two, three, four, five, six, seven of or preferably all of CD11b, CD11c, CD14, CD45, HLAII, CD31, CD34 and CD133 are used in order to confirm the absence of said markers in the selected cells; and monoclonal antibodies against one, two, three, four, of or preferably all of CD9, CD44, CD54, CD90 and CD105 are used in order to confirm the presence thereof or detectable expression levels of, at least one of and preferably all of, said markers. Said monoclonal antibodies are known, commercially available or can be obtained by a skilled person in the art by conventional methods.

IFN-γ-inducible IDO activity in the selected cells can be determined by any suitable conventional assay. For example, the selected cells can be stimulated with IFN-γ and assayed for IDO expression; then conventional Western-blot analysis for IDO protein expression can be performed and IDO enzyme activity following IFN-γ stimulation of the selected cells can be measured by tryptophan-to-kynurenine conversion with for example via High Performance Liquid Chromatography (HPLC) analysis and photometric determination of kynurenine concentration in the supernatant as the readout. Since the ASC express IDO under certain conditions, any suitable technique which allows the detection of IDO activity following IFN-γ stimulation may be used for selecting the ASC. The amount of IDO produced depends on the number of cells per square centimetre, which is preferably at a level of 5000 cells/cm² or more, but not limited to this concentration and the concentration of IFN-gamma, which ideally is 3 ng/ml or more, but not limited to this concentration. The activity of IDO produced under the described conditions will result in a detectable production of kynurenine in the micro M range after 24 hours or more.

The capacity of the selected cells to differentiate into at least two cell lineages can be assayed by conventional methods as known in the art.

ASC can be clonally expanded, if desired, using a suitable method for cloning cell populations. For example, a proliferated population of cells can be physically picked and seeded into a separate surface (or the well of a multi-well plate). Alternatively, the cells can be subcloned onto a multi-well plate at a statistical ratio for facilitating placing a single cell into each well (e.g., from about 0.1 to about 1 cell/well or even about 0.25 to about 0.5 cells/well, such as 0.5 cells/well). Of course, the cells can be cloned by plating them at low density (e.g., in a Petri dish or other suitable substrate) and isolating them from other cells using devices such as a cloning rings. The production of a clonal population can be expanded in any suitable culture medium. In any event, the isolated cells can be cultured to a suitable point when their developmental phenotype can be assessed.

It has been shown that ex vivo expansion of the ASC without inducing differentiation can be accomplished for extended time periods for example by using specially screened lots of suitable serum (such as fetal bovine serum or human serum). Methods for measuring viability and yield are known in the art (e. g., trypan blue exclusion).

Any of the steps and procedures for isolating the cells of the cell population of the invention can be performed manually, if desired. Alternatively, the process of isolating such cells can be facilitated and/or automated through one or more suitable devices, examples of which are known in the art.

In a further aspect the present invention provides ASC prepared according to the method of the invention, hereinafter said cells shall be referred to as “cells of the invention”.

COMPOSITIONS OF THE INVENTION The present invention also provides a composition comprising of the cells of the invention. Particularly preferred is a cell composition comprising essentially of the cells of the invention. Accordingly in one aspect the present invention provides a composition or population of cells wherein at least wherein at least about 50%, at least about 60%, at least about 70%, at least about 80%, at least about 85 %, at least about 90%, at least about 95% or preferably at least about 96%, 97%, 98% or 99% of the cells of said population are cells of the invention. In one embodiment said cell composition is a cell culture and accordingly further comprises suitable medium, buffers, growth factors, nutrients and/or suchlike. Said cell culture may be contained within a suitable vessel and maintained in a constant and suitable environment. Methods for the culture of cells are known in the art.

USE OF CELLS OF THE INVENTION The cells of the invention can be used for preventing, treating or ameliorating one or more symptoms associated with disease conditions. These include but are not limited to wound healing, tissue damage, allergic response, immune disease, autoimmune disease, immunologically mediated diseases, inflammatory disease, chronic inflammatory disease. Said use constitutes an additional aspect of the present invention.

Thus, in another aspect, the cells of the invention are used as a medicament. In a particular embodiment, medicaments comprising of the cells of the invention may be used for inducing transplantation tolerance, or for treating, and thereby alleviating, symptoms of autoimmune or inflammatory disorders, or immunologically mediated diseases including rejection of transplanted organs and tissues, in a subject suffering from any of said disorders or diseases. Thus, the cells of the invention can be used to therapeutically or prophylactically treat and thereby alleviate symptoms of immune, autoimmune or inflammatory disorders in a subject suffering from any of said disorders or to alleviate symptoms of immunologically mediated diseases in a subject suffering from said diseases. The cells of the invention are of use in the treatment of autoimmune disease, inflammatory disorder or immunological mediated disease. Illustrative, non-limiting examples of said diseases and disorders which can be treated are those previously listed under heading “Definitions”. In a particular embodiment, said inflammatory disease is a chronic inflammatory disease, such as, e.g., Celiac Disease, Multiple Sclerosis, Psoriasis, IBD or RA. In another aspect, the present invention relates to the use of the cells of the invention for the preparation of a medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Thus, the invention further refers to the use of the cells of the invention for the preparation of a medicament for suppressing the immune response, or for inducing transplantation tolerance, or for treating autoimmune diseases, or for treating inflammatory disorders. Examples of said autoimmune diseases and inflammatory diseases have been previously mentioned. In a particular embodiment, disease is an inflammatory disease, such as a chronic inflammatory disease, e g, Celiac Disease, Multiple Sclerosis, Psoriasis, IBD or RA.

PHARMACEUTICAL COMPOSITIONS The present invention provides pharmaceutical compositions for the treatment, prophylaxis, and amelioration of one or more symptoms associated with a disorder in which modulation of a subject's immune system is beneficial. These include autoimmune diseases, inflammatory disorders, and immunologically mediated diseases including rejection of transplanted organs and tissues.

Thus, in another aspect, the invention relates to a pharmaceutical composition, comprising cells of the invention and a pharmaceutical carrier. In a further aspect the present invention provides a pharmaceutical composition comprising ASC and/or cells of the invention, a DPP-4 antagonist or inhibitor and a pharmaceutical carrier. In one embodiment said antagonist or inhibitor is selected from the group consisting of Aminomethylpyridine; NVP DPP728; PSN9301; Isoleucine thiazolidide; Denagliptin; Sitagliptin; Vildagliptin; Saxagliptin; Alogliptin; Diprotin A. In a particularly preferred embodiment said antagonist or inhibitor is Diprotin A.

The pharmaceutical compositions of the invention comprise a prophylactically or therapeutically effective amount of one or more prophylactic or therapeutic agents (i. e. ASC and/or cells of the invention, alone or in combination with a DPP-4 antagonist or inhibitor), and a pharmaceutical carrier.

The effective amount depends on the dosage unit form, the route of administration and on other factors known in the art.

Suitable pharmaceutical carriers are known in the art and are preferably those approved by a regulatory agency of the US Federal or a state government or listed in the US or European Pharmacopeia, or other generally recognized pharmacopeia for use in animals, and more particularly in humans. The term “carrier” refers to a diluent, adjuvant, excipient, or vehicle with which the therapeutic agent is administered. The composition, if desired, can also contain minor amounts of pH buffering agents. Examples of suitable pharmaceutical carriers are described in “Remington's Pharmaceutical Sciences” by E W Martin. Such compositions will contain a prophylactically or therapeutically effective amount of a prophylactic or therapeutic agent preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, solid, semi-solid, and liquid dosage forms, such as lyophilized preparations, liquids solutions or suspensions, injectable and infusible solutions, etc. The preferred form depends on the intended mode of administration and therapeutic application.

In a further aspect the present invention provides a method for treating a subject in need thereof by administering cells of the invention or a pharmaceutical composition as disclosed herein in an amount effective to prevent, treat or ameliorate one or more symptoms associated with disease conditions.

In a further aspect the present invention provides a method for treating a subject in need thereof by administering cells of the invention and/or ASC in an amount effective to prevent, treat or ameliorate one or more symptoms associated with disease conditions and additionally administering a DPP-4 antagonist or inhibitor. In one embodiment said antagonist or inhibitor is selected from the group consisting of Aminomethylpyridine; NVP DPP728; PSN9301; Isoleucine thiazolidide; Denagliptin; Sitagliptin; Vildagliptin; Saxagliptin; Alogliptin; Diprotin A. In a particularly preferred embodiment said antagonist or inhibitor is Diprotin A. Said ASC and/or cells of the invention may be administered concurrently, contiguously, or separately to said DPP-4 antagonist or inhibitor. The effective amount depends on the dosage unit form, the route of administration and on other factors known in the art.

The administration of the cells of the invention, or the pharmaceutical composition of the invention, to the subject in need thereof can be carried out by conventional means. In a particular embodiment, said cell population is administered to the subject by a method which involves transferring the cells to the desired tissue, either in vitro (e g, as a graft prior to implantation or engrafting) or in vivo, to the animal tissue directly. The cells can be transferred to the desired tissue by any appropriate method, which generally will vary according to the tissue type. For example, cells can be transferred to a graft by bathing the graft (or infusing it) with culture medium containing the cells. Alternatively, the cells can be seeded onto the desired site within the tissue to establish a population. Cells can also be administered systemically e.g. by means of infusion of a cell suspension. Cells can be transferred to sites in vivo using devices such as catheters, trocars, cannulae, stents (which can be seeded with the cells), etc.

The cell populations and pharmaceutical compositions of the invention can be used in a combination therapy. In a specific embodiment, the combination therapy is administered to a subject with an inflammatory disorder that is refractory to one or more anti-inflammatory agents. In another embodiment, the combination therapy is used in conjunction with other types of anti-inflammatory agents including, but not limited to, nonsteroidal anti-inflammatory drugs (NSAIDs), steroidal anti-inflammatory drugs, beta-agonists, anticholingeric agents, and methyl xanthines. Examples of NSAIDs include, but are not limited to, Ibuprofen, celecoxib, diclofenac, etodolac, fenoprofen, Indomethacin, ketoralac, oxaprozin, nabumentone, suhndac, tolmentin, rofecoxib, naproxen, ketoprofen, nabumetone, etc. Such NSAIDs function by inhibiting a cyclooxgenase enzyme (e g, COX-I and/or COX-2). Examples of steroidal anti-inflammatory drugs include, but are not limited to, glucocorticoids, dexamethasone, cortisone, hydrocortisone, prednisone, prednisolone, triamcinolone, azulf[iota]dine, and eicosanoids such as thromboxanes, and leukotrienes. Monoclonal antibodies, such as Infliximab, can also be used.

In accordance with the above embodiment, the combination therapies of the invention can be used prior to, concurrently or subsequent to the administration of such anti-inflammatory agents. Further, such anti-inflammatory agents do not encompass agents characterized herein as lymphoid tissue inducers and/or immunomodulatory agents.

In another aspect, the present invention relates to the use of the cells of the invention for the preparation or manufacture of a pharmaceutical composition or medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues.

In a further aspect, the present invention relates to the use of the cells of the invention and/or ASC in combination with a DPP-4 antagonist or inhibitor for the preparation or manufacture of a pharmaceutical composition or medicament for preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues. Said antagonist or inhibitor is preferably selected from the group consisting of Aminomethylpyridine; NVP DPP728; PSN9301; Isoleucine thiazolidide; Denagliptin; Sitagliptin; Vildagliptin; Saxagliptin; Alogliptin; Diprotin A. It is particularly preferred that said DPP-4 antagonist or inhibitor is Diprotin A.

Thus, the invention further refers to the use of the cells of the invention either alone or in combination with a DPP-4 antagonist or inhibitor as well as a DPP-4 antagonist or inhibitor in combination with ASC for the preparation or manufacture of a pharmaceutical composition or medicament for suppressing the immune response, or for inducing transplantation tolerance, or for treating autoimmune diseases, or for treating inflammatory disorders.

KITS. In a further embodiment the present invention provides kits of use in treating a subject with ASC therapies. Said kit comprises i) cells of the invention or a pharmaceutical composition of the invention and ii) a device for administering said cells. Said devices include but are not limited to syringes, injection devices, catheters, trocars, cannulae and stents.

In a further embodiment, kits of the invention may further comprise instructions for use in the treatment of a subject.

USES The methods, cells, pharmaceutical compositions and kits of the present invention may be used in preventing, treating or ameliorating one or more symptoms associated with disease conditions, in which modulation of a subject's immune system is beneficial. These include autoimmune diseases, inflammatory disorders, and immunologically mediated diseases including rejection of transplanted organs and tissues. These include but are not limited to tissue damage, allergic response, immune disease, autoimmune disease, immunologically mediated diseases, inflammatory disease, chronic inflammatory disease. Specific examples of such diseases are disclosed in the “definitions” section. Said use constitutes an additional aspect of the present invention.

The invention is further described in the following examples, to which the present invention is by no means limited.

### EXAMPLES

### Example 1

In order to demonstrate the effectiveness of DPP-4 inhibition in increasing ASC cytokine or chemokine mediated mobility, ASC were induced to migrate by exposure to SDF-1 (a CXCR4 ligand that induces chemotaxis) in the presence of Diprotin A (Ile-Pro-Ile)-an inhibitor of DPP-4.

ASC were obtained from lipoaspirates obtained from human adipose tissue from healthy adult donors. Lipoaspirates were washed twice with PBS, and digested at 37° C. for 30 minutes with 0.075% collagenase (Type I, Invitrogen, Carlsbad, Calif.) in PBS. The digested sample was washed with 10% of fetal bovine serum (FBS), treated with 160 mM CINH4, suspended in culture medium (DMEM containing 10% FBS) and filtered through a 40-μm nylon mesh. Cells were seeded (2-3×10⁴ cells/cm²) onto tissue culture flasks and expanded at 37° C. and 5% CO₂, changing the culture medium every 7 days. Cells were passed to a new culture flask (1,000 cells/cm²) when cultures reached 90% of confluence. Cells were phenotypically characterized by their capacity to differentiate into chondro-, osteo-, and adipo-genic lineages. A pool of 6 ASC samples from different donors (culture passage 4-6) was used.

ASC (10⁴ cells) were seeded onto the upper surface of a Transwell permeable support insert with a pore size of 8 μm. The permeable supports were inserted into a 24 well plate. After overnight culture (37° C. and 5% CO₂) the upper surface of the inserts was treated with Diprotin A (0, 1, 5, 10 mM), and SDF-1 (50 μM) was added into the well to induce migration of the ASC to the underside of the permeable surface.

After 24 h, the upper surface of the insert was gently scraped to remove cells that did not migrate, and the inserts were stained with 0.9% crystal violet (diluted in 10% ethanol) and washed. Stained cells (i.e. those having migrated to the underside of the permeable support) were then counted. Total number of counted cells per experimental condition is shown (two inserts per condition were counted).

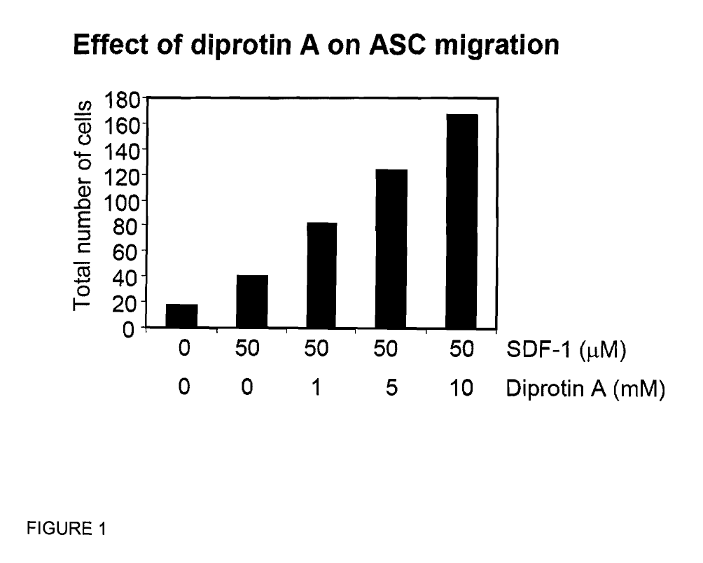

As can be seen in FIG. 1, ASC migration to the underside of the permeable support increased in proportion to the amount of Diprotin A applied to the upper surface of the support. Accordingly it can be concluded that Diprotin A exposure increases the migration of ASC in response to a SDF-1 gradient.

1.-17. (canceled)

18. A method for the preparation of adipose stem cells comprising of exposing said adipose derived stem cells to a CD26 antagonist or inhibitor.

19. A method according to claim 18 wherein said antagonist or inhibitor is selected from the group consisting of Aminomethylpyridine; P32/98; NVP DPP728; PSN9301; Isoleucine thiazolidide; Denagliptin; Sitagliptin; Vildagliptin; Saxagliptin; Alogliptin; Diprotin A.

20. A method according to claim 18 wherein said adipose derived stem cells have been previously expanded.

21. A method according to claim 18 wherein said cells are passaged at least one or more times.

22. Cells obtainable according to the method of claim 18.

23. A pharmaceutical composition comprising of the cells of claim 22 and a pharmaceutically acceptable carrier.

24. A pharmaceutical composition comprising of adipose derived stem cells, a CD26 antagonist or inhibitor and a pharmaceutically acceptable carrier.

25. A kit comprising of i) cells according to claim 22 and ii) a device for administering said cells.

26. A kit comprising of i) a pharmaceutical composition according to claim 23 and ii) a device for administering said cells.

27. A kit comprising of i) a pharmaceutical composition according to claim 24 and ii) a device for administering said cells.

28. A method for preventing, treating, or ameliorating a disease or disorder in a subject comprising of: administering to said subject cells according to claim 22 in an amount effective to prevent, treat or ameliorate said disease or disorder in said subject.

29. The method of claim 28 wherein said cells are administered at one or more In-vivo locations in need of prevention, treatment or amelioration of said disease or disorder.

30. The method of claim 28 wherein said disease or disorder is selected from the group consisting of tissue damage, allergic response, immune disease, autoimmune disease, immunologically mediated diseases, inflammatory disease, chronic inflammatory disease.

31. A method for preventing, treating, or ameliorating a disease or disorder in a subject comprising of: administering to said subject a pharmaceutical composition according to claim 23 in an amount effective to prevent, treat or ameliorate said disease or disorder in said subject.

32. A method of claim 31 wherein said cells are administered at one or more in-vivo locations in need of prevention, treatment or amelioration of said disease or disorder.

33. The method of claim 31 wherein said disease or disorder is selected from the group consisting of tissue damage, allergic response, immune disease, autoimmune disease, immunologically mediated diseases, inflammatory disease, chronic inflammatory disease.

34. A method for preventing, treating, or ameliorating a disease or disorder in a subject comprising of: administering to said subject adipose derived stem cells in an amount effective to prevent, treat or ameliorate said disease or disorder in said subject and a CD26 antagonist or inhibitor.

35. The method of claim 34 wherein said CD26 antagonist or inhibitor is administered to said subject concurrently, contiguously, or separately from said adipose derived stem cells.

36. The method of claim 34 wherein either or both of said cells or said CD26 antagonist or inhibitor are administered at one or more in-vivo locations in need of prevention, treatment or amelioration of said disease or disorder.

37. The method of claim 34 wherein said disease or disorder is selected from the group consisting of tissue damage, allergic response, immune disease, autoimmune disease, immunologically mediated diseases, inflammatory disease, chronic inflammatory disease.
